(19) United States
(12) Patent Application Publication
(10) Pub. No.: US 2004/0024425 A1
(43) Pub. Date: Feb. 5, 2004

(54) Method and apparatus for using a cardiac stimulating, sensing and guidewire combination

(76) Inventors: Seth J. Worley, Lancaster, PA (US); Paul A. Kurth, Rancho Palos Verdes, CA (US)

Correspondence Address: Daniel L. Dawes, MYERS, DAWES & ANDRAS LLP, 19900 MacArthur Blvd, Ste 1150, Irvine, CA, 92612, US

(21) Appl. No.: 10/289,726

(22) Filed: Nov. 7, 2002

Related U.S. Application Data
(60) Provisional application No. 60/399,812, filed on Jul. 31, 2002.

(51) Int. Cl. A61N 1/37

(52) U.S. Cl. 607/27

(57) ABSTRACT

A conductive guidewire having at least an exposed distal tip is endovascularly steered into an epicardial surface of a heart via the venous tributaries of the coronary sinus. A selected site is contacted by the tip of the guidewire and tested by means of the guidewire to determine suitability for permanent lead implantation. Implantation of the lead at the contacted site is contingent on the determination of the adequacy of all electrophysiological parameters. Testing includes the steps of determining the amplitude and slew rate of the local electrogram, generating an electrical stimulus, communicating the stimulus to the contacted site by means of the guidewire, sensing an electrophysiological response to the stimulus through the guidewire and determining that the pacing threshold parameters are appropriate. A permanent pacing lead is then chosen and guided by means of the guidewire to the contacted site and implanted.

### RELATED APPLICATIONS

The present application is related to U.S. Provisional Patent Application, serial No. 60/399,812, filed on Jul. 31, 2002, which is incorporated herein by reference and to which priority is claimed pursuant to 35 USC 119.

### BACKGROUND OF THE INVENTION

1. Field of the Invention

The invention relates to the field of cardiac guidewires and sensing and pacing catheters.

2. Description of the Prior Art

Baeten et.al., “Method And Apparatus For Optimum Positioning Of A Muscle Stimulating Implant” U.S. Pat. No. 5,425,751 (1995) describes an insulated conductor 36 with sheath 42 with an exposed electrode 44. The electrode 44 is to be implanted in the muscle tissue for stimulation purposes. The connector 38 is adapted to be coupled to one of the output terminals of the pulse generator after the electrode 44 has been implanted into the muscle tissue. Prior to implanting the electrode 44, it is desirable to determine the optimum implant location. This reference describes the method for making this determination. This is done by positioning the distal end of a test electrode such as a surgical needle 32 to be in contact with various test locations on the surface of the muscle. A temporary conductor wire 10 is provided for supplying test electrical current to the test probe 32. The outside surface of the gripping portion of needle 32 is spaced from the sharp muscle-contacting probe and is provided with a suitable insulating coating 32a to prevent current leakage from the needle 32. The distal end 32b of the probe must make electrical contact with the muscle tissue and therefore is not insulated.

Bourgeois et.al., “Minimally Invasive Medical Electrical Lead” U.S. Pat. No. 5,716,392 (1998) is similar to Baeten and describes medical electrical leads which may be implanted into a body organ or tissue and used for electrical stimulation of body tissue to treat various pathological conditions. Referring to the Figures, the lead body 61 consists of a conductor 6 with an insulative sleeve 65. Lead body 61 is attached to electrode 59 and to a needle 53. A test wire 69 is also attached to needle 53 and comprises a conductor and insulative sheath. At the end of test wire 64, is connected a test pen 71 so that needle 53 may be temporarily connected to a pulse generator. Needle 53 may then be touched against the heart tissue at various locations so as to determine the electrical characteristics of the local area of the heart tissue in order to make the necessary preliminary determinations.

Benzing III et.al., “Method And Apparatus For Measuring The Ohmic Contact Resistance Of An Electrode Attached To Body Tissue” U.S. Pat. No. 4,245,643 (1981) describes an electrode attached to a body tissue, for example a cardiac tissue, and a pacemaker electrode surgically implanted in the heart. Based on the measured contact resistance, the surgeon is able to select an optimum low-resistance location at which to implant the electrode in the heart.

Gielen, “System And Method For Optimized Brain Stimulation” Pub. No. US2001/0008972 (2001) describes an object of the invention to determine with a test lead or a permanent lead when a brain electrode is positioned properly so that during stimulation of the brain target a desired result is obtained. In order to accomplish this, the invention provides for a test lead system with conventional DBS electrodes which are introduced, and by varying the position of the electrodes, observing the relevant body patient movement or other reaction and thus determining the optimum position for the location of the electrodes before the permanent leads are introduced.

Chachques et.al., “Method And Apparatus Including A Sliding Insulation Lead For Cardiac Assistance” U.S. Pat. No. 4,735,205 (1988) is directed to leads provided with electrode surface areas which can be varied at the time of surgical implantation to reflect a desired length of exposed electrode surface area extending through the muscle. The portion of electrode 44, which is left exposed may be adjusted by sliding tubing 42 axially over the insulating sleeve 36 and exposed electrode 44 in a direction away from the connector 38. As the sliding tube 42 is withdrawn, the conductive electrode 44 is exposed and the exposed length is indicated by the distance between the exterior marker 45 and the interior marker 48.

What is needed is a device and method for testing the response of heart muscle or any tissue to an electrical probe prior to implanting a sensing and stimulating lead at the tested site, which probe is also used to guide or implant the lead.

### BRIEF SUMMARY OF THE INVENTION

The invention is a method and apparatus to pretest the actual electrophysiological functioning of an anatomically determined pacing site prior to permanent lead implantation by use of a pacing and sensing wire and then to use that same wire as a guidewire for implantation.

The purpose of the pretest may be for any cardiac function, but is expressly contemplated as including optimal sensing and biventricular pacing or resynchronization, and avoidance of inadvertent diaphragmatic pacing.

The invention contemplates the use of a pacing wire for use in the heart and or its epicardial venous system having a diameter of approximately 0.014″-0.018″, which wire is insulated along its length except for a proximal portion of approximately 1-10 cm and a distal portion of approximately 1-5 mm. The wire is placed into the heart or its venous system. First electrogram characteristics and timing are determined and then current applied to it to allow the physician to determine whether the wire, which will be used as a guide for a pacemaker lead, is evoking the optimal response desired. The wire position may also help determine which of several permanent pacing leads is to be deployed.

Traditionally a pacemaker lead is advanced to the desired position in the heart and then the sensing characteristic and electrophysiological responses to pacing are determined. After initial lead placement it is very common that the site chosen for lead placement turns out not to be advantageous for various reasons. Low sensed voltage or high pacing threshold due to myocardial scarring is a common problem, the site of stimulation causes diaphragmatic pacing directly or via the phrenic nerve, or the desired improvement in left ventricular resynchronization is not accomplished. Having a way of easily and rapidly testing multiple sites for suitable pacing and sensing greatly improves the results accomplished over the current methods. Use of a fine, insulated wire allows for the site chosen for the pacing lead to be tested for any undesired result before the pacemaker lead is implanted as well as to determine the optimum permanent pacing lead which is to be employed.

In addition use of the sensing wire allows for optimal biventricular pacing, i.e. allows the physician to find the place where the electrical impulse pulses arrives last in the posterior lateral wall of the left ventricular as compared to the surface electrocardiogram. It allows the physician to see the activation of a test site relative to the surface QRS duration, or the entirety of right and left ventricular depolarization pulse. This then allows the lateral wall of the left ventricle to be paced by the same insulated wire and to determine what affect this may have on the pacing of the left ventricular posterior lateral wall to achieve more normal QRS duration as well as resynchronization of the left ventricular contraction which has been altered by left bundle branch block and dilated cardiomyopathy. Thereafter, the wire is used as a guidewire for the lead implantation at the optimized resynchronization site.

The invention is particularly characterized by the use of the same instrument which serves to sense electrical wave forms and stimulate the heart or tissue at multiple test sites, as the guidewire by which a temporary or permanent sensing or stimulating lead is implanted into the heart or tissue.

More particularly, the invention is a method comprising the steps of endovascularly steering a conductive guidewire having at least an exposed distal tip into a heart chamber; contacting a selected site within or on the surface of the heart with the tip of the guidewire; testing the contacted site to determine suitability for lead implantation by means of the guidewire; guiding a lead by means of the guidewire to the contacted site; and implanting the lead at the contacted site. Epicardial surface contact is made via the heart's venous system. The venous system is accessed via the coronary sinus, which is accessed from the right atrium.

In one embodiment, the step of testing comprises testing the contacted site to determine that the chosen site does not result in diaphragmatic stimulation either directly or via the phrenic nerve.

In another embodiment the step of testing comprises testing the contacted site to determine timing of the local ventricular activation relative to the totality of the a QRS complex wavefront at the contacted site. A site is located which is activated very late during the QRS complex. Pre-exciting this area will dramatically shorten the QRS duration of the paced beat and result in much better resynchronization. In another embodiment, the step of testing is the determination of the local electrogram characteristics in terms of signal amplitude and dV/dT or slew rate, i.e. the time rate of change of the electrical signal. The total QRS duration is determined by the total amount of time required for all the ventricular myocardium to be depolarized. The local chosen site is tested for signal adequacy for sensing. This electrogram may last only 20-40 msec while the total QRS duration is greater than 130 msec. As the depolarization wave approaches the electrode, it is positive. As it passes the electrode there is a rapid transition to a negative wave. This rapid transition reflects the local activation time and the slew rate helps to determine the adequacy of the signal. The pacing threshold is determined by stimulating the site with pulses of a known duration (usually 0.5 msec) and then decrementing the voltage in gradual steps until a response is no longer evoked. The lowest pulse amplitude in voltage and current which still evokes a response is called the pacing or stimulation threshold.

In another embodiment, the step of testing is the determination of stimulation threshold. The adequacy of the site for pacing is determined by the stimulation threshold. The stimulation threshold is the voltage and current required to evoke a response. The voltage and current also define the impedance of the site. All these parameters are determined for all pacing sites used in pacing therapy. In terms of sensing, the peak-to-peak amplitude of the recorded signal is measured in millivolts. Adequate signals are generally those measured to be in excess of the sensing threshold of commonly used pacemakers.

In another embodiment the step of testing comprises testing the contact site for adequacy in the sensing parameters, e.g. optimal changes in simultaneously recorded left ventricular time rate of change of pressure, Dp/Dt. In an optimal test site the change in blood pressure over time of the left ventricular cavity is optimized before lead implantation. What is measured is the first derivative of the pressure tracing. This first derivative is a measure of the velocity by which the ventricle contracts. In general, the step of testing comprises testing the contacted site to determine all the an electrophysiological parameters of the contacted site.

The step of implanting the lead at the contacted site is contingent on determination of the electrophysiological parameters of the contacted site. Again what this means is that the parameters required for long term sensing and pacing are appropriate for currently used pacemakers. In other words, that the signals are of sufficient amplitude to be sensed and that the energy required to pace the site is sufficiently low so that battery drain will not be excessive.

The step of guiding the lead by means of the guidewire to the contacted site comprises telescopically disposing the lead over the guidewire while the guidewire is left in place at the contacted site or using the wire as a conduit for lead placement.

The step of testing can also be characterized as comprising the steps of generating an electrical stimulus; determining electrical resynchronization; communicating a stimulus to the contacted site by means of the guidewire; measuring an electrophysiological response to the stimulus confirming mechanical resynchronization and implanting a lead by means of the guidewire while the guidewire remains in place at the contact site of stimulus and sensing.

The invention is also directed to an apparatus for guiding the implantation of a lead or catheter comprising a steerable, conductive guidewire; an exposed distal tip defined on the conductive guidewire; an exposed proximal portion defined on the conductive guidewire; and insulation disposed on the conductive guidewire between the distal tip and the proximal portion.

In one embodiment the apparatus further comprises an electronic circuit coupled to the guidewire for generating an electrical stimulus. This is generally performed by attaching a lead or wire to what is referred to as a pacing system analyzer (PSA). These are sophisticated temporary pacemakers where the local event can be measured in terms of voltage and timing, and a pacing threshold can be easily determined. Such pacemakers are conventional with each pacemaker manufacturer, and any temporary pacemaker can be employed to make these measurements.

In another embodiment the apparatus further comprises an electronic circuit coupled to the guidewire for sensing an electrophysiological signal coupled to the guidewire at its distal tip.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a cut-away diagrammatic side view of a heart into which a sensing guidewire of the invention has been steered and contacted to a resynchronization site on the epicardial surface on the lateral wall of the left ventricle via a venous tributary of the coronary sinus.

FIG. 2 is the cut-away diagrammatic side view of FIG. 1 in which a pacemaker lead has been implanted using the sensing guidewire just before the removal of the sensing guidewire and connection of the lead to a pacemaker.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

A conductive guidewire having at least an exposed distal tip is endovascularly steered into a heart chamber or preferably to the epicardial surface of the heart via the venous tributaries of the coronary sinus. A selected site is contacted within or on the surface the heart by the tip of the guidewire. The contacted site is tested by means of the guidewire to determine suitability for permanent lead implantation. The contacted site may be tested to determine that inappropriate diaphragmatic stimulation is not present, to determine timing of the local activation time relative to the duration of the QRS complex, or to determine any electrophysiological parameter of the contact site. Implantation of the lead at the contacted site is contingent on determination of the adequacy of all electrophysiological parameters. Testing includes the steps of determining the amplitude and slew rate of the local electrogram, generating an electrical stimulus, communicating the stimulus to the contacted site by means of the guidewire, sensing an electrophysiological response to the stimulus through the guidewire and determining that the pacing threshold parameters are appropriate. A permanent pacing lead is then chosen and guided by means of the guidewire to the contacted site and implanted.

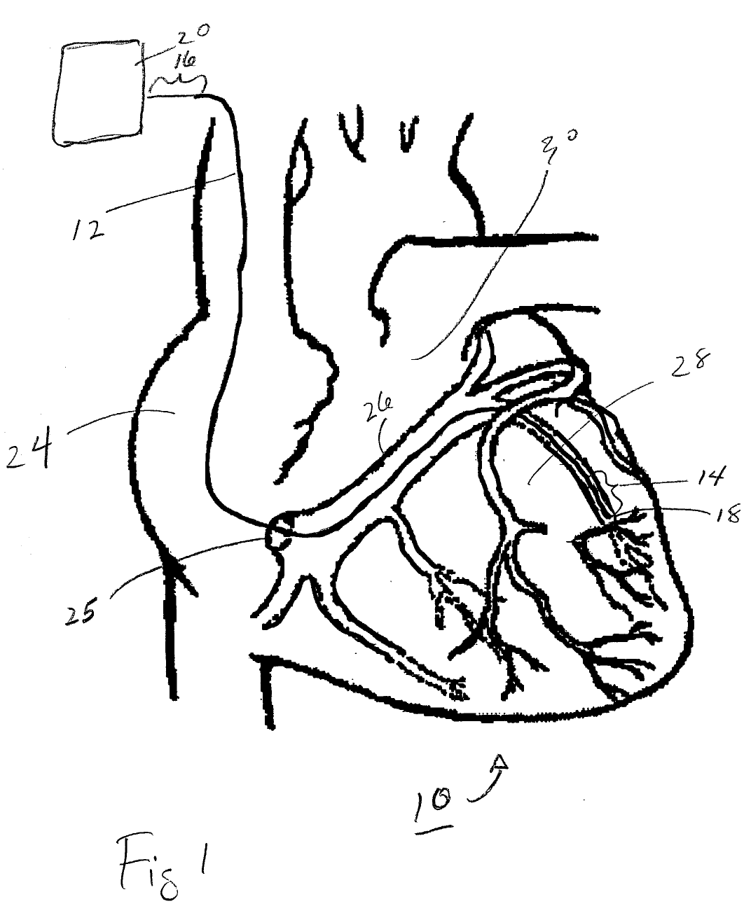

FIG. 1 is a diagrammatic depiction of a heart 10 into which a sensing guidewire 12 has been endovascularly inserted. Sensing guidewire 12 is comprised of a conductive elongate member or wire, which is insulated except for a distal-most portion 14 and a proximal portion 16, which are left uninsulated. Distal-most portion 14 is uninsulated so that it can be used as a test probe for sensing the electrical signals in the heart muscle to which tip 18 is touched or inserted. Proximal portion 16 is uninsulated so that it can be used for electrical connection to external conventional electronics 20 used to detect and analyze the heart signal on guidewire 12 and/or to place a test signal or stimulating pulse or pulses to evoke a desired cardiac response.

Guidewire 12 may be insulated using any medical polymer now known or later devised. In the preferred embodiment 1 to 10 mm of tip 18 is uninsulated, although the amount of exposure can be varied according to well understood design principles without departure from the spirit and scope of the invention. Approximately, 1 to 10 cm of proximal portion 16 is uninsulated in the illustrated embodiment, although as it can be readily appreciated the amount of exposure of proximal portion 16 is not usually critical. In general, guidewire 12 is thin enough, even when insulated, to allow for sufficient flexibility for manipulation through the vascular system into the heart chambers. The diameter of guidewire 12, including insulation, is in the range of 0.014-0.018 inch, although other ranges may be acceptable depending on the composition of guidewire 12.

On the other hand, guidewire 12 may have sufficient memory and stiffness so that it can be precurved by the physician and steered by conventional means when tip 18 is disposed in the heart chambers. In the illustration of FIG. 1 guidewire 12 is shown as being endovascularly inserted through the right atrium 24 into the coronary sinus 25 and one of its venous tributaries 26 to place tip 18 into contact with a postero-lateral epicardial area of the left ventricle 28. In the illustration, this is for the purpose of placing a second pacing lead in contact with the posterior wall of left ventricle 28 to resynchronize ventricle 28, whose electromechanical activation has been altered by left bundle branch block and dilated cardiomyopathy.

In such operations, it may be difficult to determine whether or not the contact regions selected for tip 18 might electrically communicate with the diaphragm either directly or via the phrenic nerve (not shown) located just below heart 10. The application of a test signal generated in electronics 20 and communicated by guidewire 12 to the contact site in heart 10 allows the physician to determine whether or not such inappropriate stimulation has inadvertently been made. If it has, tip 18 can be easily withdrawn and relocated to a different site and retested until it is empirically certain that no unintended electrical connection to the diaphragm has been made.

Still further, even when an appropriate contact site for tip 18 has been found relative to the diaphragmatic stimulation, it is advantageous to be able to empirically confirm that the contact site has the electrophysiological characteristics desired such as optimal electrical resynchronization and optimal left ventricular pressure development Dp/Dt, i.e. the time rate of pressure change. For example, in the case of left ventricular and biventricular pacing, it is advantageous to pace the site in postero-lateral left ventricular area where the myocardial activation last arrives during the QRS complex. This can only be determined by measuring the arrival time of the local activation at various sampled sites relative to a surface EKG or other timing standard. Sensing guidewire 12 can be steered by the physician to different sites to map out the arrival time of the local wavefront in the lateral wall of the left ventricle 22 and to determine the optimal site for pacing and sensing.

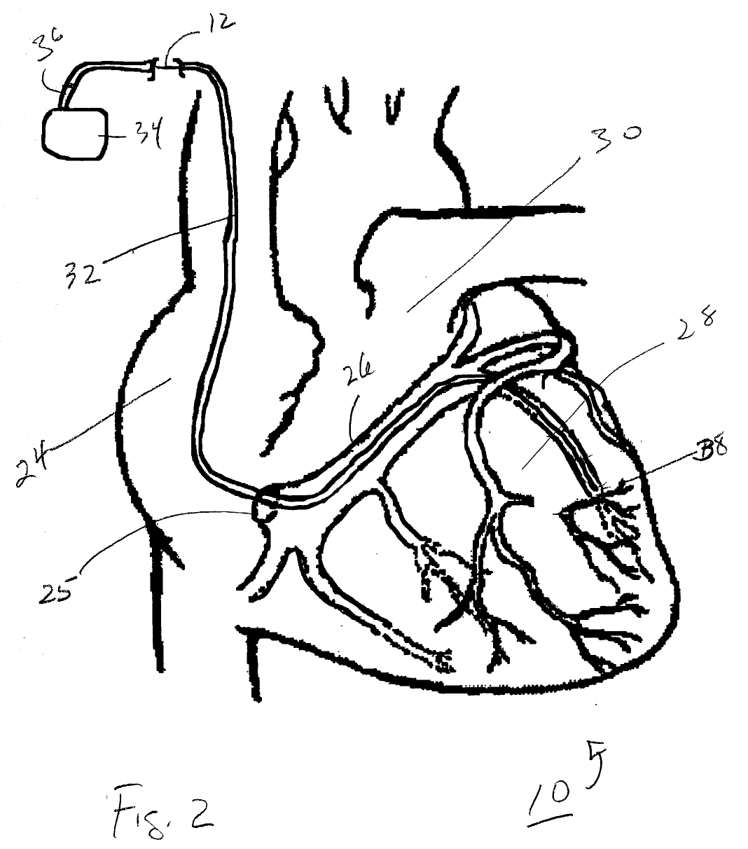

Once on optimal site for contact has thus been sensed through guidewire 12, guidewire 12 is left in place and used as the guiding wire for pacemaker lead implantation at the optimized resynchronization site. FIG. 2 diagrammatically illustrates a pacemaker lead 32 telescopically disposed over sensing guidewire 12 after tip 18 has been located at the optimized resynchronization site. A portion of pacemaker lead 32 is cutaway to show sensing guidewire 22 axially disposed in a lumen defined in pacemaker lead 32. Pacemaker lead 32 is then implanted by conventional means at the optimized resynchronization site for the delivery of left ventricle pacing stimuli through pacemaker 34 which is typically subcutaneously implanted at the proximal end of lead 32 after guidewire 12 is removed and connected to connector 36 on lead 32. The tip 38 of pacemaker lead 32 is thus unerringly led by sensing guidewire 12 to the optimized resynchronization site.

While the illustrated embodiment has been described in terms of left ventricular resynchronization, it is to be expressly understood that the invention can be practiced in a similar manner in any electrocardial operation according to the teachings of the invention.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. A method comprising:
endovascularly placing a conductive guidewire having at least an exposed distal tip into or on an epicardial surface of a heart;
contacting a selected cardiac site with the tip of the guidewire;
testing the contacted site to determine suitability for lead implantation by means of the guidewire;
guiding a lead by means of the guidewire to the contacted site; and
implanting the lead at the contacted site.

2. The method of claim 1 where testing the contacted site comprises testing the contacted site to determine that diaphragmatic stimulation does not occur either directly or via the phrenic nerve.

3. The method of claim 1 where testing the contacted site comprises testing the contacted site to determine timing of the local myocardial activation relative to the total duration of the QRS complex.

4. The method of claim 1 where testing the contacted site comprises testing the contacted site to determine the amplitude and slew rate of the local electrogram.

5. The method of claim 1 where testing the contacted site comprises testing the contacted site to determine the electrophysiological parameters of the contacted site in terms both of sensing and pacing characteristics

6. The method of claim 5 where implanting the lead at the contacted site is contingent on determination of the adequacy of the electrophysiological parameters of the contacted site.

7. The method of claim 1 where guiding the lead by means of the guidewire to the contacted site comprises telescopically disposing the lead over the guidewire while the guidewire is left in place at the contacted site.

8. The method of claim 1 where guiding the lead by means of the guidewire to the contacted site comprises using the wire, which is at least partially coupled with the lead to place the lead in the desired location.

9. The method of claim 1 where testing the contacted site comprises:
sensing and timing of a local myocardial depolarization event;
generating an electrical stimulus;
communicating the stimulus to the contacted site by means of the guidewire; and
determining a pacing threshold by measuring an electrophysiological response to the stimulus.

10. The method of claim 1 where implanting a lead by means of the guidewire while the guidewire comprises disposing the lead with the use of the guidewire, while the guidewire remains in place at the contact site of stimulus and sensing.

11. An apparatus for guiding the implantation of a lead or catheter comprising:
a steerable, conductive guidewire and;
an exposed distal tip defined on the conductive guidewire;
an exposed proximal portion defined on the conductive guidewire; and
insulation disposed on the conductive guidewire between the distal tip and the proximal portion.

12. The apparatus of claim 11 further comprising an electronic circuit coupled to the guidewire for generating an electrical stimulus.

13. The apparatus of claim 11 further comprising an electronic circuit coupled to the guidewire for sensing an electrophysiological signal coupled to the guidewire at its distal tip.

14. The apparatus of claim 12 further comprising an electronic circuit coupled to the guidewire for sensing an electrophysiological signal coupled to the guidewire at its distal tip.
